(19) United States
(12) Patent Application Publication
(10) Pub. No.: US 2025/0366737 A1
(43) Pub. Date: Dec. 4, 2025

(54) ImpactGuard

(71) Applicant: Aharon Bar-Gill, ZICHRON-YAAKOV (IL)

(72) Inventor: Aharon Bar-Gill, ZICHRON-YAAKOV (IL)

(21) Appl. No.: 19/221,426

(22) Filed: May 28, 2025

Related U.S. Application Data
(60) Provisional application No. 63/654,881, filed on May 31, 2024.

(51) Int. Cl. A61B 5/11; A61B 5/00

(52) U.S. Cl. CPC A61B 5/1117; A61B 5/6804

(57) ABSTRACT

The present disclosure provides a wearable fall protection device comprising a harness configured to be worn by a user, a plurality of telescopic rod modules attached to the harness, each telescopic rod module comprising an outer housing, a telescopic rod initially located within the outer housing and rod movement elements configured to move the telescopic rod, a plurality of sensors configured to detect a fall of the user, and a processor configured to receive data from the sensors, determine if the user is falling based on the received data, and activate deployment of at least one of the telescopic rod modules if the user is determined to be falling.

### CROSS-REFERENCE TO RELATED APPLICATIONS

This application claims priority from U.S. Provisional Patent Application No. 63/505,046 filed May 30 2024, which is hereby incorporated by reference in its entirety.

### FIELD OF INVENTION

The present disclosure relates to wearable fall protection devices, and more particularly to a wearable device with deployable telescopic rods for mitigating injuries from falls.

### BACKGROUND

Falls among older adults and individuals with certain medical conditions are a significant public health concern. These incidents can lead to serious injuries, reduced mobility, and decreased quality of life. As the global population ages, the prevalence of fall-related injuries is expected to increase, placing greater strain on healthcare systems worldwide.

Traditional fall prevention strategies often focus on environmental modifications, exercise programs, and medication management. However, these approaches may not provide immediate protection during an actual fall event. There is a growing interest in developing wearable technologies that can actively intervene to reduce the impact of falls when they occur.

Existing wearable fall protection devices face several challenges. Many are bulky or uncomfortable, limiting user compliance. Some rely on inflatable systems, which can be complex and may involve safety concerns such as fast high-temperature burning or high-pressure gas. Others use rigid structures that, while protective, can restrict normal movement and may be socially stigmatizing for users.

Sensor technologies for fall detection have improved in recent years, but accurately distinguishing between normal activities and fall events remains difficult. False positives can lead to unnecessary deployments, while false negatives may leave users unprotected during actual falls. Additionally, the diverse range of fall scenarios and individual user characteristics further complicates the development of reliable fall detection algorithms.

Energy absorption during falls is another area of ongoing research. Effective fall protection requires dissipating kinetic energy to reduce impact forces on the body. However, designing compact, wearable systems capable of managing these forces without causing secondary injuries is challenging.

As the field of wearable fall protection evolves, there is a need for innovative solutions that address these limitations. Ideally, such devices would be unobtrusive, comfortable for extended wear, and capable of rapid, targeted intervention during fall events. Improving sensor accuracy, energy absorption mechanisms, and overall system integration could enhance the effectiveness of wearable fall protection technologies and potentially reduce the incidence of fall-related injuries.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a wearable fall protection device is provided. The device includes a harness configured to be worn by a user. The device includes a plurality of telescopic rod modules attached to the harness, each telescopic rod module comprising an outer housing, a telescopic rod initially located within the outer housing and rod movement elements configured to move the telescopic rod. The device includes a plurality of sensors configured to detect a fall of the user. The device includes a processor configured to receive data from the sensors, determine if the user is falling based on the received data, and activate deployment of at least two of the telescopic rod modules if the user is determined to be falling.

According to other aspects of the present disclosure, the device may include one or more of the following features. The plurality of sensors may comprise at least one laser range finder sensor and at least one inertial measurement unit. The processor may be further configured to determine a direction of the fall based on data from the plurality of sensors. The processor may be configured to selectively activate deployment of specific telescopic rod modules based on the determined direction of the fall. Each telescopic rod module may further comprise a torsion spring configured to initiate deployment of the telescopic rod module and a solenoid configured to release the torsion spring upon activation by the processor. Each telescopic rod module may further comprise a safety ball attached to an end of the telescopic rod, the safety ball configured to contact a surface during deployment. The processor may be further configured to perform a personalized training process to establish fall detection parameters specific to the user and adjust the fall detection parameters in real-time based on ongoing sensor measurements.

According to another aspect of the present disclosure, a method for protecting a user from fall injuries is provided. The method includes detecting, using a plurality of sensors, motion data of a user wearing a harness with attached telescopic rod modules, each telescopic rod module comprising an outer housing, a telescopic rod initially located within the outer housing and rod movement elements configured to move the telescopic rod. The method includes analyzing, using a processor, the motion data to determine if the user is falling. The method includes, if the user is determined to be falling, activating deployment of at least two of the telescopic rod modules to brake the user's fall.

According to other aspects of the present disclosure, the method may include one or more of the following features. The method may further comprise determining a direction of the fall based on the motion data from the plurality of sensors. Activating deployment of at least two of the telescopic rod modules may comprise selectively activating specific telescopic rod modules based on the determined direction of the fall. Each telescopic rod module may comprise a torsion spring and a solenoid, and activating deployment may comprise releasing the torsion spring using the solenoid to initiate deployment of the telescopic rod module. Each telescopic rod module may further comprise a safety ball attached to an end of a telescopic rod, and the method may further comprise contacting a surface with the safety ball during deployment of the telescopic rod module. The method may further comprise performing a personalized training process to establish fall detection parameters specific to the user and adjusting the fall detection parameters in real-time based on ongoing sensor measurements. Analyzing the motion data may comprise comparing the motion data to the adjusted fall detection parameters to determine if the user is falling.

According to another aspect of the present disclosure, a non-transitory computer-readable medium storing instructions is provided. When executed by a processor, the instructions cause the processor to perform operations for a fall protection system comprising a wearable harness, a plurality of telescopic rod modules attached to the harness, each telescopic rod module comprising an outer housing, a telescopic rod initially located within the outer housing and rod movement elements configured to move the telescopic rod, one or more laser range finders, and an inertial measurement unit. The operations include receiving data from the one or more laser range finders configured to measure distances between the harness and surrounding surfaces. The operations include receiving data from the inertial measurement unit configured to detect acceleration and orientation of the harness. The operations include processing the data from the laser range finders and inertial measurement unit. The operations include determining based on the processed data if a fall is occurring. The operations include triggering extension of at least two telescopic rod module if a fall is determined to be occurring.

According to other aspects of the present disclosure, the non-transitory computer-readable medium may include one or more of the following features. The telescopic rod modules may each comprise an outer tube, a telescopic rod telescopically housed within the outer tube, a torsion spring configured to initiate extension of the telescopic rod, and a solenoid configured to release the torsion spring upon activation. Each telescopic rod module may further comprise a safety ball attached to an end of the telescopic rod, the safety ball configured to contact a surface during extension of the telescopic rod module. The operations may further comprise performing a personalized training process to establish fall detection parameters specific to a user and adjusting the fall detection parameters in real-time based on ongoing measurements from the laser range finders and inertial measurement unit. Determining if a fall is occurring may comprise comparing data from the laser range finders and inertial measurement unit to the adjusted fall detection parameters. The operations may further comprise determining a direction of the fall based on the data from the laser range finders and inertial measurement unit and selectively triggering extension of specific telescopic rod modules based on the determined direction of the fall.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a kinematic diagram depicting fall detection and braking concepts, according to aspects of the present disclosure.

FIG. 2 depicts a system diagram of a wearable harness assembly with component labels, according to an embodiment.

FIG. 3 shows a detailed view of a telescopic rod module assembly and its attachments, in accordance with example embodiments.

FIG. 4 presents a sectional view of a telescopic rod assembly with internal components, according to aspects of the present disclosure.

FIG. 5 illustrates a flowchart showing a process for fall detection and braking activation, according to an embodiment.

FIG. 6 illustrates a flowchart showing a process for fall detection and braking activation, according to an embodiment.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

According to an embodiment there is provided a method for reducing the impact of falls. The method includes:

Determining (using one or more laser distance sensors that measures one or more rates of one or more change of one or more distances along one or more directions between a region of a body of a person and the surface as well as using acceleration sensors for measuring one or more accelerations of the region of the body of the person along the one or more directions) whether a person is falling. The determining is based on a distinction between movement of the person that do not amount to falling and an expected movement of the person when falling. The distinction may be tailored to the person—for example by using a machine learning process.

The region of the body is located above the center of gravity of the person.

The harness may carry multiple laser distance sensors and multiple rate gyro sensors (packaged in IMSs along with acceleration sensors) located at different locations to allow a determination of the direction of the fall.

The respond may be relevant to different situations—for example walking or running on a relatively flat surface, climbing stairs or descending stairs, reaching a wall, facing one or more obstacles and the like.

When a falling event is detected-two or more telescopic rods are moved away from the body while mechanically coupled to a harness worn by the person and have an interfacing mechanism connected to their edges which contact the surface. In order to prevent a significant force spike aimed vertically at the chest of the person due to the contact, the algorithm controls the beginning instant of the deployment. Consequently, the angle between the telescopic rod and the surface at touchdown is determined. Its value avoids vertical-to-body force push and assures slide-out of the truncated ball.

The present disclosure relates to a wearable fall protection device designed to mitigate injuries resulting from falls. In some cases, the device may comprise a harness configured to be worn by a user. The harness may include multiple telescopic rod modules attached at various points. Each telescopic rod module may include an outer housing, a telescopic rod, and rod movement elements. These components may work together to enable controlled deployment of the rods when needed.

The device may also incorporate multiple sensors designed to detect a user's fall. These sensors may continuously monitor the user's movement and position. In some cases, the device may include a processor configured to receive and analyze data from the sensors. The processor may be programmed to determine if a fall is occurring based on the sensor data. Upon detecting a fall, the processor may activate the deployment of one or more telescopic rod modules.

The deployment of the telescopic rod modules may serve to brake or slow the user's fall, potentially reducing the risk of serious injury. By combining wearable hardware with sensor technology and intelligent processing, the device may provide a proactive approach to fall protection.

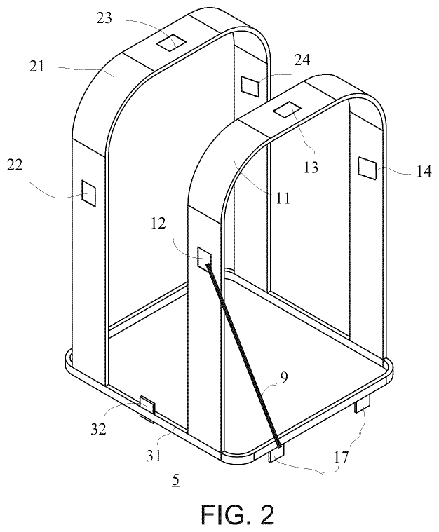

In some cases, the wearable fall protection device may include a harness assembly configured to be worn by a user. FIG. 2 illustrates an example of such a harness assembly. The harness assembly may comprise a left strengthened strap 11 and a right strengthened strap 21. These strengthened straps 11, 21 may form the main structural elements of the harness assembly.

The left strengthened strap 11 and the right strengthened strap 21 may be connected by a horizontal back strap. This horizontal back strap may provide additional stability and support to the harness assembly.

In some cases, the harness assembly may include a belt 31 that extends across the lower portion of the assembly. The belt 31 may be secured by a belt buckle 32 at the front of the harness assembly. The belt 31 and belt buckle 32 may provide a means for securing the harness assembly around the wearer's body.

The harness assembly may incorporate multiple laser range finder (LRF) sensors and inertial measurement units (IMUs) at different locations-see sensor modules 13 and 23. These sensors may be positioned to monitor distances between the harness and surrounding surfaces, as well as to detect acceleration and orientation of the harness. The sensor modules may be located at any other location—for example at the front of the harness—at the rear of the harness—at both rear and front of the harness, and the like. The sensor module may be located so that the range sensors are oriented slightly in front and/or slightly back to the person—or slightly tilted to the side of the person (slightly—for example may be at a tile of 5-20 degrees in relation to the horizon).

In some cases, the harness assembly may include an electric strap for channeling power and data. This electric strap may connect various components of the harness assembly, such as the sensors and other electronic elements.

The wearable fall protection device may also include a mannequin for storage, charging, and donning/doffing of the harness assembly. This mannequin may provide a convenient means for storing the harness assembly when not in use, as well as facilitating the process of putting on and taking off the harness. The mannequin may also incorporate charging capabilities, allowing the device's battery to be recharged while stored.

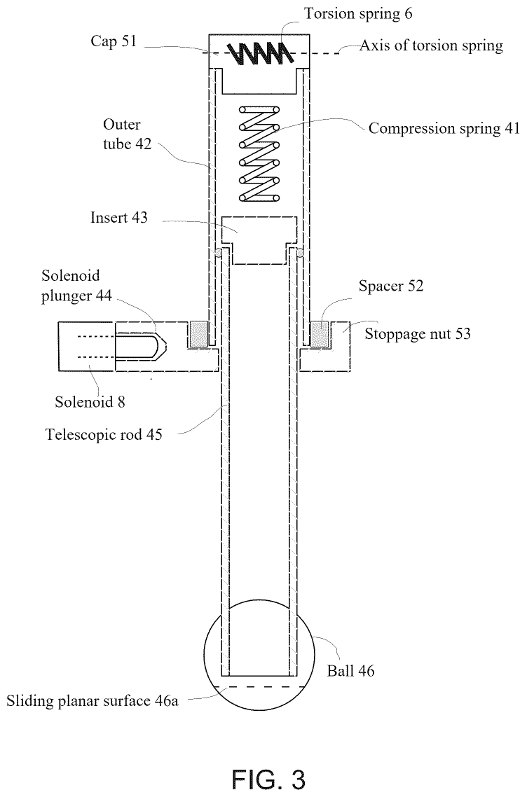

In some cases, the wearable fall protection device may include one or more telescopic rod module assemblies. FIG. 3 illustrates a detailed view of a telescopic rod module assembly and its attachments.

The telescopic rod module assembly may include an upper support connected to a telescopic rod. The telescopic rod 3 may comprise an outer tube 4 and a telescopic rod 5. In some cases, the telescopic rod 45 may be telescopically housed within the outer tube 4, allowing for extension when deployed.

A torsion spring 6 may be housed within the assembly to facilitate deployment of the telescopic rod module. The torsion spring 6 may be configured to initiate deployment of the telescopic rod module when activated. In some cases, the telescopic rod module assembly may also include a push-out spring in addition to the torsion spring 6 to further assist in the deployment process.

The lower portion of the assembly may include a lower support (detached to the belt) that may be configured to mount to the belt of the harness system. A solenoid 8 may be integrated into the lower support 17 to control deployment of the telescopic rod module. The solenoid 8 may be configured to release the torsion spring 6 upon activation by a processor, initiating the deployment process.

At the bottom end of the telescopic rod 45, a ball 42 may be attached to provide a controlled interface with the ground surface during deployment. In some cases, the ball 42 may have a segment sliced off to ensure slide-out upon surface contact. This feature may help manage contact forces and enable the sliding motion needed for energy absorption during a fall event.

The telescopic rod module assembly may be designed for one-time use only. After deployment, the entire telescopic rod module may need to be replaced to ensure proper functionality for future fall protection.

The components of the telescopic rod module assembly may be arranged to enable both the angular deployment away from the body and the telescopic extension needed for fall arrest functionality. Multiple telescopic rod modules may be mounted to the harness system at various positions to provide fall protection in different directions.

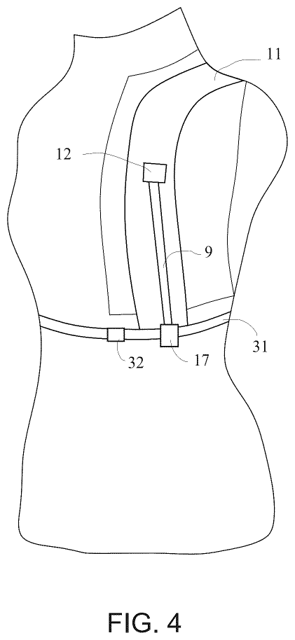

In some cases, the telescopic rod assembly may include additional internal components to facilitate controlled deployment and energy absorption during a fall event. FIG. 4 illustrates a sectional view of a telescopic rod assembly, revealing the internal arrangement of components.

The telescopic rod assembly may include an outer tube 42 that houses several internal components. At the upper portion of the assembly, a cap 51 may be positioned at the top. In some cases, a compression spring 41 may be located beneath the cap 51. The compression spring 41 may provide additional force for deployment or assist in energy absorption during extension.

An insert 43 may be positioned within the outer tube 42. The insert 43 may serve to guide the movement of internal components or provide structural support within the assembly.

The telescopic rod assembly may include a solenoid plunger 44 that interacts with the internal mechanisms. The solenoid plunger 44 may be configured to release or activate other components within the assembly upon receiving a signal from a processor.

A telescopic rod 45 may be housed within the outer tube 42, allowing for telescopic extension. In some cases, the telescopic rod 45 may be designed to extend smoothly from the outer tube 42 when deployed.

At the lower end of the telescopic rod 45, a ball 46 may be attached to provide a contact interface. The ball 46 helps to manage contact forces and enable sliding motion during deployment.

The telescopic rod assembly may also incorporate a spacer 52 and a stoppage nut 53 that help secure and position components within the assembly. The spacer 52 may provide proper spacing between components, while the stoppage nut 53 may be used to fasten or adjust the positioning of internal elements. The telescopic rod assembly also includes a stopper to keep the upper end of the extension within the outer housing.

In some cases, the components of the telescopic rod assembly may be arranged in a manner that enables controlled telescopic extension when activated. The overall height of the assembly may vary depending on the specific application and user requirements.

The telescopic rod assembly may be designed to work in conjunction with other components of the fall protection device, such as the torsion spring 6 and the solenoid 8, to provide effective fall protection and energy absorption during deployment.

An example of a distance between the cap and the stoppage nut is about 25 centimeters, and the distance between th insert and the ball is about 25 centimeter-other dimensions may be provided.

In some cases, the wearable fall protection device may include a plurality of sensors configured to detect a fall of the user. FIG. 2 illustrates a system diagram showing components of a wearable harness assembly, including sensors for fall detection.

The sensors may include at least one laser range finder sensor. In some cases, the harness assembly may incorporate a left distance sensor 13 and a right distance sensor 23. The left distance sensor 13 and the right distance sensor 23 may be laser range finder sensors configured to measure distances between the harness and surrounding surfaces. These distance sensors may be positioned on the left and right sides of the harness assembly to provide comprehensive spatial awareness.

In addition to the laser range finder sensors, the device may include at least one inertial measurement unit (IMU). The IMU may be configured to detect acceleration and orientation of the harness. In some cases, the IMU may be integrated into the harness assembly alongside the distance sensors.

The wearable fall protection device may include a processor configured to receive and process data from the sensors. The processor may analyze the data from the laser range finder sensors and the inertial measurement units to determine if a fall is occurring. In some cases, the processor may generate instantaneous obstacle maps of the surroundings based on the sensor data. These obstacle maps may provide real-time information about the user's environment, which may be used in fall detection algorithms.

The processor may be programmed to determine the direction of the fall based on the sensor data. By analyzing the measurements from the left distance sensor 13 and the right distance sensor 23, along with data from the IMU, the processor may calculate the trajectory and orientation of the user during a potential fall event.

In some cases, the processor may use a combination of distance measurements and acceleration data to distinguish between normal movements and fall events. The processor may compare the sensor data to pre-established thresholds or patterns indicative of a fall. If the sensor data exceeds these thresholds or matches fall patterns, the processor may trigger the deployment of the telescopic rod modules.

The control system may be designed to minimize false positives while ensuring rapid response to actual fall events. In some cases, the processor may employ machine learning algorithms to improve fall detection accuracy over time, adapting to the user's specific movement patterns and environmental conditions.

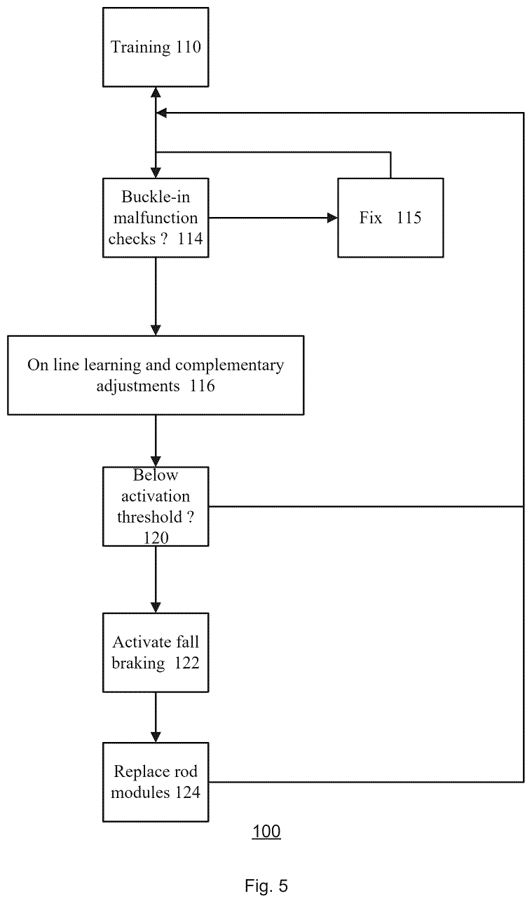

In some cases, the wearable fall protection device may implement a process for fall detection and rod deployment. FIG. 5 illustrates a flowchart showing this process.

The process may begin with a training step 110. During this step, the processor may perform a personalized training process to establish fall detection parameters specific to the user. This training may involve collecting sensor data while the user performs various movements and activities. The processor may analyze this data to establish baseline patterns and thresholds for normal movement.

Following the training step 110, the process may move to a buckle-in malfunction checks step 114. This step may involve verifying the proper functioning of various components of the device, such as the sensors and telescopic rod modules. If a malfunction is detected, the process may proceed to a fix step 115, where the issue may be addressed before continuing.

After the malfunction checks, the process may continue to an on-line learning and complementary adjustment step 116. During this step, the processor may adjust the fall detection parameters in real-time based on ongoing sensor measurements. This continuous learning process may allow the device to adapt to changes in the user's movement patterns or environmental conditions over time.

The process may then move to a decision point 120, where the processor may determine if measurements are below an activation threshold. This step may involve detecting motion data of a user wearing the harness with attached telescopic rod modules using the plurality of sensors. The sensors may include the left distance sensor 13, the right distance sensor 23, and an inertial measurement unit.

If the measurements are below the activation threshold, indicating a potential fall, the process may proceed to step 122 where the system may be activated for fall braking. This activation may involve deploying one or more of the telescopic rod modules. The deployment may be initiated by the solenoid 8 releasing the torsion spring 6, which may cause the telescopic rod 45 to extend from the outer tube 42.

After activation and deployment, the process may move to step 124 where the telescopic rod module may be replaced. This step may be necessary as the telescopic rod modules may be designed for one-time use.

The flowchart shows multiple feedback paths. After the telescopic rod module replacement step 124, the process may return to the training step 110. This loop may allow the system to recalibrate and adjust its parameters after each deployment event. Similarly, after the fix step 115, the process may also return to the training step 110, ensuring that any resolved malfunctions do not affect the system's performance.

This process may enable the wearable fall protection device to continuously monitor the user's movements, quickly detect potential falls, and deploy the appropriate telescopic rod modules to mitigate fall-related injuries.

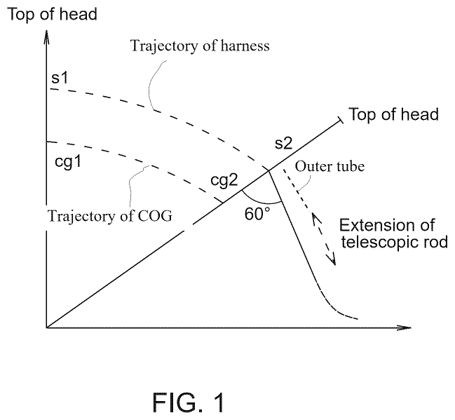

In some cases, the wearable fall protection device may operate through a coordinated interaction of multiple components to detect falls and deploy telescopic rods for impact mitigation. FIG. 1 illustrates a kinematic diagram depicting the concept of fall detection and braking. The diagram shows an initial gravity point g1, representing the user's position before a fall. As a fall occurs, the user's position may change to a final gravity point g2.

The harness assembly, as shown in FIG. 2, may include multiple components working together to detect and respond to falls. The left strengthened strap 11 and right strengthened strap 21 may form the main structural elements of the harness. These straps may support various interfaces and sensors. A front left rod interface 12 and a left rear rod interface 14 may be attached to the left strengthened strap 11, while a front right rod interface 22 and a right rear rod interface 24 may be attached to the right strengthened strap 21. These interfaces may serve as connection points for the telescopic rod modules.

The sensor modules 13 and 23 may be positioned between the respective rod interfaces on each side. These sensors may be laser range finders configured to measure distances between the harness and surrounding surfaces. The harness assembly may also lower support which may be used for supporting elements such as the solenoid and other elements, which may provide mounting points for solenoids used in the rod deployment mechanism.

In some cases, the fall protection system may comprise the wearable harness, a plurality of telescopic rod modules attached to the harness, one or more laser range finders, and an inertial measurement unit. The inertial measurement unit may be integrated into the harness assembly to detect acceleration and orientation changes.

FIG. 3 illustrates a detailed view of a telescopic rod module assembly. The upper support may connect to the telescopic rod, which comprises the outer tube 42 and the telescopic rod 45. The torsion spring 6 may be housed within the assembly to facilitate deployment. The lower support 17 of FIG. 2 may mount to the belt of the harness system and may integrate the solenoid 8 for controlling deployment. The safety ball 9 at the bottom end of the telescopic rod 45 may provide a controlled interface with the ground surface during deployment.

The internal components of the telescopic rod assembly are shown in FIG. 4. The outer tube 42 may house several components, including the cap 51, the compression spring 41, the insert 43, the solenoid plunger 44, and the telescopic rod 45. The ball 46 at the lower end of the telescopic rod 45 may be partially truncated (see sliding planar surface 46a illustrating a truncated edge of the ball) serves to interface with the surface and allow the sliding of the telescopic rod following the contact with the surface. The spacer 52 and the stoppage nut 53 may help secure and position components within the assembly.

The sequence of events from fall detection to impact mitigation may follow the process illustrated in FIG. 5. The process may begin with a training step 110, where fall detection parameters specific to the user may be established. After malfunction checks in step 114 and potential fixes in step 115, the system may engage in online learning and complementary measurements in step 116.

In some cases, the processor may selectively activate specific telescopic rod modules based on the fall direction. The processor may analyze data from the laser range finders and inertial measurement units to determine if measurements are below an activation threshold in step 120. If a fall is detected, the system may be activated for fall braking in step 122.

Upon activation, the solenoid 8 may release the torsion spring 6, initiating the deployment of the telescopic rod 45. The telescopic rod 45 may extend from the outer tube 42, with the ball 46 providing a controlled interface with the ground surface. This deployment may serve to brake or slow the user's fall, potentially reducing the risk of serious injury.

After deployment, the telescopic rod module may be replaced in step 124, as the modules may be designed for one-time use. The process may then loop back to the training step 110, allowing the system to recalibrate and adjust its parameters after each deployment event.

A number of implementations have been described. Nevertheless, it will be understood that various modifications may be made without departing from the spirit and scope of the disclosure. Accordingly, other implementations are within the scope of the following claims.

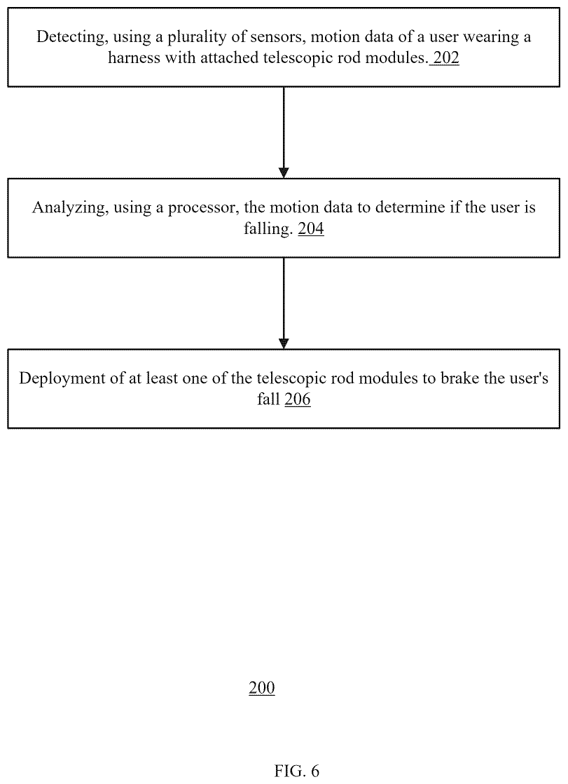

FIG. 6 illustrates a method 200 for protecting a user from fall injuries according to an embodiment of the present disclosure. The method 200 begins with detecting 202, using a plurality of sensors, motion data of a user wearing a harness with attached telescopic rod modules. Each telescopic rod module comprises an outer housing, a telescopic rod initially located within the outer housing and rod movement elements configured to move the telescopic rod.

The method 200 continues with analyzing 204, using a processor, the motion data to determine if the user is falling. This analysis may include comparing the motion data to personalized fall detection parameters established for the specific user.

If the user is determined to be falling, the method 200 proceeds with activating 206 deployment of at least two of the telescopic rod modules to brake the user's fall. The method 200 may further include determining 208 a direction of the fall based on the motion data from the plurality of sensors, and selectively activating 210 specific telescopic rod modules based on the determined direction of the fall.

Examples of calculations are provided below. The values provided below are merely non-limiting examples of values.

ImG functional flow builds on our Base Model of a person, who may begin falling in whatever direction from standing stance or walking gait.

It's structured after the Free-Fall Model, adapted for the Ground Reaction Force (GRF) effect and parameter-tuned via numerous and random-Initial-Conditions-in-course of Full-Scale Development.

Regarding the statistical properties of the ImG solution and following the guidelines in Medicept.com (Risk Management Series—Part 6 “Estimating Probability of Occurrence,” Oct. 15, 2020), probability <10⁻⁵ is required for preventing false alarms, both negative and positive.

We use two independent sensor technologies, LRFs and IMUs. The joint error probability (Gaussian) is the product of probabilities. With 3σ-level errors for each sensor, the threshold value is 1.8*10⁻⁶. As shown in the sequel, the requirement is satisfied.

### GRF Model—Incl Typical Numeric Example

Before falling GRF=a_{z_before_fall}=a_{z_fall_0}=10 m/sec². It begins decreasing linearly (approximate) right away. From our fall experiments (corroborated by published results), the jerk (Δa_{z_fall}/Δt)=const (value about 10 m/sec³).

$$Δ {a}_{z} = [ ( {a}_{z \_ fall \_ 0} - ( Δ {a}_{z \_ fall} / Δ t ) Δ t ] = 10 m / {sec}^{2} - 10 m / {sec}^{3} * t$$

$${V}_{z} = [ ( {a}_{z \_ fall \_ 0} * t - 0.5 ( Δ {a}_{z \_ fall} / Δ t ) {t}^{2} = [ 10 m / {sec}^{2} * t - 5 m / {sec}^{3} * {t}^{2} ]$$

$$h = {h}_{0 \_ candidate} - [ ( 0.5 * {a}_{z \_ fall \_ 0} ) {t}^{2} - 1 / 2 * ( Δ {a}_{z \_ fall} / Δ t ) ( 1 / 3 ) {t}^{3} ] = {h}_{0 \_ candidate} - [ 5 {t}^{2} - 5 / 3 {t}^{3} ]$$

$$Δ h = [ 5 {t}^{2} - 5 / 3 {t}^{3} ]$$

Note that the above model's first term is the classic Free Fall model.

For Δt_{Decision}=160 msec and the above value for (Δa_{z_fall}/Δt), at the end of this interval we get the Pseudo-Free-Fall vertical travel Δh=[(5 m/sec²t²−(5/3) m/sec³t³)]=12.0 cm.

$$For Δ {t}_{end \_ of \_ unfolding} = 400 msec , we obtain Δ h = 70 cm .$$

$$Uncertainties - at Δ {t}_{Decision} = 160 msec we have Δ h = 12. cm$$

For model uncertainty (4σ_{Δh}=20% to be on the safe side), 4σ_{Δh}=2.4 cm.

For LRFs, 2%−error for measuring height is 3σ level and shoulders height of 120 cm, 1σ=0.8 cm. Mean error over 6 LRFs, e.g., 1σ=0.8 cm/6^{1/2}=0.33 cm, yielding 4σ=1.4 cm. 4σ_{Δh} RSS=2.8 cm.

To cross a lower bound=120 cm of the walking band, with error probability of 10⁻⁵, we need to measure: 120 cm-2.8 cm=117.2 cm (4σ_{Δh}).

### Candidate Fall Instants

Since fall-beginning instant is a priori unknown, we refer to each sampled instant (16 msec apart) as a candidate-fall-beginning instant.

Through the walking-gait phase, shoulders, perform periodic vertical displacements within a band of up to 3 cm upwards and 5 cm downwards.

Sampling these height measurements within the walking band, we may detect either up-stepping or monotonous increase in Δh and in Δa_{z}; the latter may be either falling or down-stepping. If their signs are reversed, we have up-stepping and there is no falling.

If a candidate is sampled on the Upper Bound of the walking Band, downward crossing of the Lower Bound triggers the Activation Decision; the unfolding command is issued within the next few tens of milliseconds.

In case the fall beginning point is on the Lower Bound of the walking band, the deployment command will be issued at Δt_{Decision}=160 msec & Δh=12 cm i.e. 12 cm below the crossing.

We apply the algorithm from sample at t=0.096 msec relative to the corresponding candidate. The five checks are at 0.096 msec, 0.112 msec, 0.128 msec, 144 msec, 160 msec. The decision is taken right after t=0.160 msec, to allow for the activation of the solenoid and the two springs.

After each sample, the algorithm works on 5 strings of sampled data.

Δt each time sampling point & for n LRFs, h′ₘₑₐₙ and σ_{h′mean}=relative to this mean are computed. We refer to the instantaneous measured/computed values of h′ₘₑₐₙ as candidate beginning-of fall values.

We use h′ₘₑₐₙ and σ_{h′mean} of minima of walking periods to obtain hₘₑₐₙ and σₕₘₑₐₙ of the walking Lower Bound.

Standing Stance Scenario-Physical variables vis-à-vis sensors with their accuracies

IMU: GRF=const=g, angular rate components=0, accel horizontal components=0.

MEMS accelerometers' accuracy (non-exotic) is 3σ=5 mg. Since for Δa_{z}/Δt we need to subtract a_{z} values, 1σ=2^{1/2}(0.05/3)=0.023 m/sec².
LRF: Δh=0

Obtaining Δ-values requires subtraction, 1σ*2^{1/2}. For 1σ_Δh_{LRF}=0.33 cm, we get: 1σ_Δh=2^{1/2}*0.33=0.5 cm.

Condition (a) Δh, with 2σ=1.0 cm: —probability that in a standing scenario, ImG will decide that the person is falling is much lower than 10⁻⁵. We thus avoid a False Positive alarm.-probability that in a falling scenario, ImG will decide that the person is standing is much lower than 10⁻⁵. We thus avoid a False Negative alarm.

Δa_{z}—with 1σ=0.023 m/sec² Condition (b):
probability that in a standing scenario, ImG will decide that person is falling is much less than 10⁻⁵, i.e., we avoid a False Positive alarm.
probability that in a falling scenario, ImG decides that person is standing is much less than 10⁻⁵ i.e., we avoid a False Negative alarm.

In principle, we need that product of error probabilities of Conditions (a) & (b) satisfies the joint error probability requirement of 10⁻⁵ (4σ). In practice, each of the two conditions alone satisfies the requirement.

Any reference to “may be” should also refer to “may not be”.

In the foregoing detailed description, numerous specific details are set forth to provide a thorough understanding of the one or more embodiments of the disclosure. However, it will be understood by those skilled in the art that the present one or more embodiments of the disclosure may be practiced without these specific details. In other instances, well-known methods, procedures, and components have not been described in detail so as not to obscure the present one or more embodiments of the disclosure.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the disclosure may for the most part, be implemented using electron optic components and circuits known to those skilled in the art, details will not be explained in any greater extent than that considered necessary as illustrated above, for the understanding and appreciation of the underlying concepts of the present one or more embodiments of the disclosure and in order not to obfuscate or distract from the teachings of the present one or more embodiments of the disclosure.

Any reference in the specification to a method should be applied mutatis mutandis to a method capable of executing the method.

Any reference in the specification to a method and any other component should be applied mutatis mutandis to a method that may be executed by a method.

Any combination of any module or unit listed in any of the figures, any part of the specification and/or any claims may be provided. Especially any combination of any claimed feature may be provided.

In the foregoing specification, the invention has been described with reference to specific examples of embodiments of the invention. It will, however, be evident that various modifications and changes may be made therein without departing from the broader spirit and scope of the invention as set forth in the appended claims.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Any reference to “consisting”, “having” and/or “including” should be applied mutatis mutandis to “consisting” and/or “consisting essentially of”.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It is appreciated that various features of the embodiments of the disclosure which are, for clarity, described in the contexts of separate embodiments may also be provided in combination in a single embodiment. Conversely, various features of the embodiments of the disclosure which are, for brevity, described in the context of a single embodiment may also be provided separately or in any suitable sub-combination.

It will be appreciated by persons skilled in the art that the embodiments of the disclosure are not limited by what has been particularly shown and described hereinabove. Rather, the scope of the embodiments of the disclosure is defined by the appended claims and equivalents thereof.

1. A wearable fall protection device, comprising:
a harness configured to be worn by a user;
a plurality of telescopic rod modules attached to the harness, each telescopic rod module comprising an outer housing, a telescopic rod initially located within the outer housing and rod movement elements configured to move the telescopic rod;
a plurality of sensors configured to detect a fall of the user; and
a processor configured to:
receive data from the sensors,
determine if the user is falling based on the received data, and
activate deployment of at least one of the telescopic rod modules if the user is determined to be falling.

2. The device of claim 1, wherein the plurality of sensors comprises at least one laser range finder sensor and at least one inertial measurement unit.

3. The device of claim 2, wherein the processor is further configured to determine a direction of the fall based on data from the plurality of sensors.

4. The device of claim 3, wherein the processor is configured to selectively activate deployment of specific telescopic rod modules based on the determined direction of the fall.

5. The device of claim 1, wherein each telescopic rod module further comprises:
a torsion spring configured to initiate deployment of the telescopic rod module; and
a solenoid configured to release the torsion spring upon activation by the processor.

6. The device of claim 5, wherein each telescopic rod module further comprises a safety ball attached to an end of the telescopic rod, the safety ball configured to contact a surface during deployment.

7. The device of claim 1, wherein the processor is further configured to:
perform a personalized training process to establish fall detection parameters specific to the user; and
adjust the fall detection parameters in real-time based on ongoing sensor measurements.

8. A method for protecting a user from fall injuries, comprising:
detecting, using a plurality of sensors, motion data of a user wearing a harness with attached telescopic rod modules, each telescopic rod module comprising an outer housing, a telescopic rod initially located within the outer housing and rod movement elements configured to move the telescopic rod;
analyzing, using a processor, the motion data to determine if the user is falling; and
if the user is determined to be falling, activating deployment of at least one of the telescopic rod modules to brake the user's fall.

9. The method of claim 8, further comprising determining a direction of the fall based on the motion data from the plurality of sensors.

10. The method of claim 9, wherein activating deployment of at least one of the telescopic rod modules comprises selectively activating specific telescopic rod modules based on the determined direction of the fall.

11. The method of claim 8, wherein each telescopic rod module comprises a torsion spring and a solenoid, and wherein activating deployment comprises:
releasing the torsion spring using the solenoid to initiate deployment of the telescopic rod module.

12. The method of claim 11, wherein each telescopic rod module further comprises a safety ball attached to an end of a telescopic rod, the method further comprising:
contacting a surface with the safety ball during deployment of the telescopic rod module.

13. The method of claim 8, further comprising:
performing a personalized training process to establish fall detection parameters specific to the user; and
adjusting the fall detection parameters in real-time based on ongoing sensor measurements.

14. The method of claim 13, wherein analyzing the motion data comprises comparing the motion data to the adjusted fall detection parameters to determine if the user is falling.

15. A non-transitory computer-readable medium storing instructions that, when executed by a processor, cause the processor to perform operations for a fall protection system comprising a wearable harness, a plurality of telescopic rod modules attached to the harness, each telescopic rod module comprising an outer housing, a telescopic rod initially located within the outer housing and rod movement elements configured to move the telescopic rod, one or more laser range finders, and an inertial measurement unit, the operations comprising:
receiving data from the one or more laser range finders configured to measure distances between the harness and surrounding surfaces;
receiving data from the inertial measurement unit configured to detect acceleration and orientation of the harness;
processing the data from the laser range finders and inertial measurement unit;
determining based on the processed data if a fall is occurring; and
triggering extension of at least one telescopic rod module if a fall is determined to be occurring.

16. The non-transitory computer-readable medium of claim 15, wherein the telescopic rod modules each comprise an outer tube, a telescopic rod telescopically housed within the outer tube, a torsion spring configured to initiate extension of the telescopic rod, and a solenoid configured to release the torsion spring upon activation.

17. The non-transitory computer-readable medium of claim 16, wherein each telescopic rod module further comprises a safety ball attached to an end of the telescopic rod, the safety ball configured to contact a surface during extension of the telescopic rod module.

18. The non-transitory computer-readable medium of claim 15, wherein the operations further comprise:
performing a personalized training process to establish fall detection parameters specific to a user; and
adjusting the fall detection parameters in real-time based on ongoing measurements from the laser range finders and inertial measurement unit.

19. The non-transitory computer-readable medium of claim 18, wherein determining if a fall is occurring comprises comparing data from the laser range finders and inertial measurement unit to the adjusted fall detection parameters.

20. The non-transitory computer-readable medium of claim 19, wherein the operations further comprise:
determining a direction of the fall based on the data from the laser range finders and inertial measurement unit; and
selectively triggering extension of specific telescopic rod modules based on the determined direction of the fall.
